(19) United States
(12) Patent Application Publication
(10) Pub. No.: US 2026/0245128 A1
(43) Pub. Date: Aug. 20, 2026

(54) METHOD AND DEVICE FOR RECOMMENDING COSMETICS SUITABLE FOR USER ON BASIS OF USER'S SKIN TYPE AND EFFICACY OF COSMETIC INGREDIENTS

(71) Applicant: LULULAB INC., Seoul (KR)

(72) Inventors: Jinhee LEE, Busan (KR); Jong Ha LEE, Hwaseong-si (KR); Sangwook YOO, Seoul (KR); Yongjoon CHOE, Seoul (KR)

(73) Assignee: LULULAB INC., Seoul (KR)

(21) Appl. No.: 18/879,019

(22) PCT Filed: Jun. 27, 2023
(86) PCT No.: PCT/KR2023/008926

§ 371 Date: Dec. 26, 2024

(30) Foreign Application Priority Data
Jun. 28, 2022 (KR) 10-2022-0079131

(51) Int. Cl. G06Q 30/0601

(52) U.S. Cl. CPC G06Q 30/0631

(57) ABSTRACT

A product recommendation server that recommends a product suitable for a user, according to various embodiments, may comprise: a skin type determination unit that acquires a face image of a user and determines the user's skin type on the basis of the acquired face image; a product efficacy determination unit that determines the product efficacy of each of products included in a product DB; a product determination unit that determines a product to be recommended to the user on the basis of the determined skin type and product efficacy; and a recommendation list provision unit that generates a recommendation list for the user on the basis of the determined product.

### TECHNICAL FIELD

The present disclosure relates to a method and a device for recommending cosmetics suitable for a user on the basis of the skin type of the user and the efficacy of cosmetic ingredients and, more particularly, to a method and a device for recommending cosmetics suitable for a user by setting different importance of each efficacy of cosmetic ingredients depending on the skin type of the user.

### BACKGROUND ART

Unless otherwise indicated herein, the matters described in this section are not prior art to the claims of this application and their inclusion in this section is not an admission that they are prior art.

Recently, as interest in beauty has increased, interest in skin care for the face of a user has increased. In particular, a skin condition measurement device is being developed that takes a picture of the facial skin of a user and analyzes various skin troubles (e.g., wrinkles, pores, acne, etc.) on the face of the user. Meanwhile, various systems that recommend cosmetics on the basis of a result from the skin condition measurement device have been disclosed.

However, a conventional method for recommending customized cosmetics on the basis of skin measurement results have either is a method in which pre-registered products are uniformly recommended according to skin type or a method in which ratings given by a group of users are aggregated into simple numerical values to make recommendations on the basis of rankings. In other words, it is a system designed to uniformly recommend preset products corresponding to skin items that have room for skin improvement on the basis of skin measurement results.

In addition, most existing cosmetic recommendation systems focus only on a user's perspective, highlighting a perspective on how to determine the skin type of the user, while not giving much consideration to ingredients and efficacy contained in each cosmetic product, or whether such ingredients and efficacy can be appropriately matched with the skin type of the user. Therefore, unlike existing customized cosmetic recommendation methods, a method is being developed to clearly determine the efficacy of numerous cosmetics and to ensure that the determined efficacy matches the skin type of a user.

### DISCLOSURE

### Technical Problem

In order to solve the above problems, the objective of the present disclosure is to provide a method and a device for measuring the skin type of a user and determining the efficacy of a cosmetic on the basis of various indicators to provide the cosmetic with optimal efficacy matching the measured skin type.

### Technical Solution

According to various embodiments, a product recommendation server for recommending a product suitable for a user includes: a skin type determination unit that obtains a facial image of a user and determines a skin type of the user on the basis of the obtained facial image; a product efficacy determination unit that determines product efficacy of each of products comprised in a product DB; a product determination unit that determines a product to be recommended to the user on the basis of the determined skin type and the determined product efficacy; and a recommendation list provision unit that generates a recommendation list for the user on the basis of the determined product.

According to various embodiments, the skin type determination unit may determine necessary efficacy required for the user on the basis of the determined skin type, the product determination unit may determine first products having the necessary efficacy from the product DB, and the product efficacy determination unit may calculate an efficacy score of each of the first products.

According to various embodiments, the product efficacy determination unit may recognize letters in ingredient images ingredient information of each of the products, extract comprised in each of the products on the basis of the recognized letters, determine a weight for each of ingredients on the basis of types of the ingredients comprised in each of the products, order of listing of the ingredients listed in each of the products, and the number of the listed ingredients, and calculate the efficacy score on the basis of the weight for each of the ingredients.

According to various embodiments, the product determination unit may determine second products with a preset score or higher among the first products as products to be recommended to the user on the basis of the calculated efficacy score of each of the products.

According to various embodiments, the product efficacy determination unit may determine the efficacy of each of the products on the basis of at least one of target efficacy targeted by a product company, ingredient efficacy based on ingredient information, and efficacy based on review analysis.

### Advantageous Effects

According to various embodiments disclosed in this document, not only can the skin type of a user be measured, but also the efficacy of a cosmetic that may be effective for the measured skin type of the user can be clearly extracted.

In addition, according to various embodiments, the efficacy of a cosmetic may be determined not only by the ingredients of the cosmetic or reviews about the cosmetic, but also by considering various factors.

In addition, various effects that are directly or indirectly identified through this document may be provided.

### DESCRIPTION OF DRAWINGS

FIG. 1 is a view illustrating a product recommendation system according to an embodiment.

FIG. 2 is a diagram illustrating the main components of a product recommendation server.

FIG. 3 is a diagram for determining the efficacy of each of products included in a product DB on the basis of at least one of company target efficacy, ingredient analysis efficacy, and review analysis efficacy, and recommending the product to a user.

FIG. 4 is a diagram illustrating the configuration of the hardware of the product recommendation server according to FIG. 1.

### MODE FOR INVENTION

The present disclosure may have various modifications and embodiments, and specific embodiments are illustrated in the drawings and described in detail in the detailed description. However, this is not intended to limit the present disclosure to a specific embodiment, but should be understood to include all modifications, equivalents or substitutes included in the spirit and technical scope of the present disclosure. In describing each drawing, similar reference numerals are used to refer to similar components.

Terms such as “first”, “second”, “A”, “B”, etc. may be used to describe various components, but such components should not be limited by such terms. The terms are used solely to distinguish one component from another. For example, without exceeding the scope of the present disclosure, the first component may be named the second component, and similarly, the second component may be named the first component. The term “and/or” includes any combination of a plurality of related described items or any one of a plurality of related described items.

When it is mentioned that a component is “connected” or “coupled” to another component, it should be understood that the component may be directly connected or coupled to the another component, but there may be other components present therebetween. On the other hand, when it is mentioned that a component is “directly connected” or “directly coupled” to another component, it should be understood that there are no other components therebetween.

The terminology used in this application is for the purpose of describing particular embodiments only and is not intended to limit the present disclosure. Singular expressions include plural expressions unless the context clearly indicates otherwise. In this application, it should be understood that terms such as “include” or “have” are intended to specify the presence of a feature, number, step, operation, component, part or combination thereof described in the specification, but do not exclude in advance the possibility of the presence or addition of one or more other features, numbers, steps, operations, components, parts or combinations thereof.

Unless otherwise defined, all terms used herein, including technical or scientific terms, have the same meanings as commonly understood by those skilled in the art to which the present disclosure belongs. Terms defined in commonly used dictionaries should be interpreted as having meanings consistent with their meanings in the context of the relevant art, and should not be interpreted in idealized or overly formal sense unless expressly defined in this application.

Hereinafter, preferred embodiments according to the present disclosure will be described in detail with reference to the attached drawings.

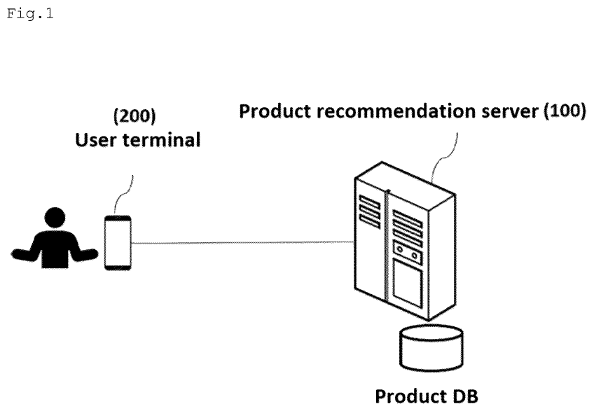

FIG. 1 is a view illustrating a product recommendation system 10 according to an embodiment. Referring to FIG. 1, the system 10 may include a product recommendation server 100, a user terminal 200, etc. Operations described below may be performed or implemented through a platform (e.g., a web page and/or an application) controlled by the server 100. In other words, the product recommendation server 100 may provide a website in which a user can input, register, and output various information by accessing the server 100 through a network by using the user terminal 200, and may provide an application installed and executed in the user terminal 200 so as to input, register, and output various information.

The product recommendation server 100 may determine the skin type of a user, determine the efficacy of each of products in a product DB, and match the determined skin type of the user with the efficacy of each of the products to determine a product to be recommended to the user. Here, the server 100 may be understood as a device separate from the user terminal 200, but the operational unit and operating configuration of the server 100 may be executed independently in the user terminal 200.

The user terminal 200 may be a desktop computer, a laptop computer, a notebook, a smart phone, a tablet PC, a mobile phone, a smart watch, a smart glass, an e-book reader, a portable multimedia player (PMP), a portable game console, a navigation device, a digital camera, a digital multimedia broadcasting (DMB) player, a digital audio recorder, a digital audio player, a digital video recorder, a digital video player, or a personal digital assistant (PDA), etc. which are capable of communication.

The product recommendation server 100 and the user terminal 200 may be each connected to a communication network to transmit and receive data between each other through the communication network. For example, as the communication network, various types of wired or wireless communication networks such as a local area network (LAN), a metropolitan area network (MAN), a global system for a mobile network (GSM), an enhanced data GSM environment (EDGE), high speed downlink packet access (HSDPA), wideband code division multiple access (W-CDMA), code division multiple access (CDMA), time division multiple access (TDMA), Bluetooth, Zigbee, Wi-Fi, voice over Internet protocol (VOIP), LTE Advanced, IEEE802.16m, WirelessMAN-Advanced, HSPA+, 3GPP long term evolution (LTE), Mobile WiMAX (IEEE 802.16e), UMB (formerly EV)-DO Rev. C), Flash-OFDM, iBurst and MBWA (IEEE 802.20) systems, HIPERMAN, beam-division n multiple access (BDMA), world interoperability for microwave access (Wi-MAX), and 5G may be used.

Throughout this specification, a case in which a product subject to analysis, recommendation, or determination is a cosmetic may be treated as a major example and described. However, the product that is subject to the analysis, recommendation or determination of the present disclosure is not limited to the cosmetic, and may refer to food, medicines and daily necessities that are composed of multiple ingredients and include labels of constituents. The cosmetic is not limited to a specific type, such as basic cosmetics, color cosmetics, fragrance cosmetics, cleansing cosmetics, and hair cosmetics.

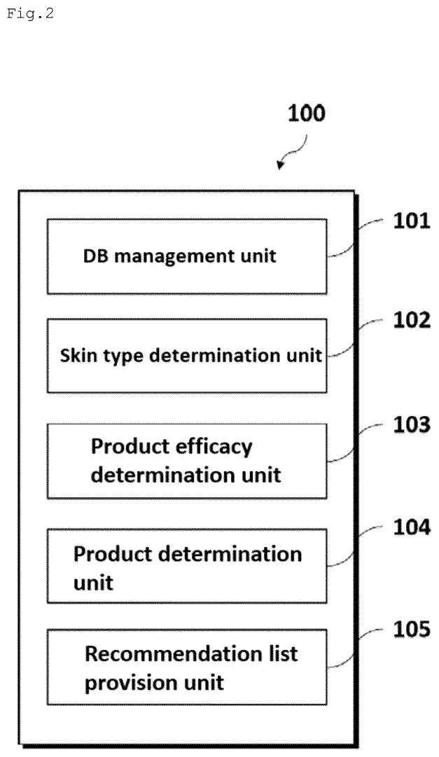

FIG. 2 is a diagram illustrating the main components of the product recommendation server 100.

The product recommendation server 100 may include a DB management unit 101, a skin type determination unit 102, a product efficacy determination unit 103, a product determination unit 104, a recommendation list provision unit 105, etc.

The DB management unit 101 may store user identification information. The user identification information may include a facial image of a user and/or voice information of a user. The DB management unit 101 may store user facial images captured through the user terminal 200 and/or a skin measurement device 300 or user facial images including user faces among images transmitted via a wired/wireless network.

The DB management unit 101 may store and manage user body information, user location information, user health information (e.g., medical information), user medical history information, etc. corresponding to an account registered in an application provided by the user terminal 200 and/or the product recommendation server 100. For example, user physical information may include height, weight, gender, skin color, and body constitution (e.g., body constitution may refer to the four constitutional types in traditional Korean medicine, such as Taeyangin, Taeumin, Soyangin, or Soeumin). User medical information may include family medical history, past medical treatment information, past diagnosis information, past surgery information, smoking history, allergy information, etc.

The DB management unit 101 may collect and store the purchase history, purchase patterns, etc. of a user (e.g., purchase time zone, cosmetics price range, cosmetics type, cosmetics name), etc. of cosmetics purchased through a cosmetics sales server (e.g., a cosmetics sales brand server) via a wired/wireless network. The cosmetics sales server may be a server that is inked with and/or collaborates with the product recommendation server 100.

The DB management unit 101 may store product information of cosmetics obtained through the cosmetics sales server. For example, the DB management unit 101 may include databases such as MongoDB and MYSQL, and may store product information acquired through websites other than the cosmetics sales server (e.g., blogs, cafes, pharmaceutical/medical institution websites) as well as the product information of cosmetics acquired through the cosmetics sales server. MongoDB may store various information such as cosmetic unique IDs, cosmetic image links, cosmetic detailed information links, cosmetic names, cosmetic prices, cosmetic ratings, the number of reviews for cosmetics, nutritional ingredients included in cosmetics, a distribution method for cosmetics, a storage method for cosmetics, and cosmetics recommended by experts. MYSQL may store product information for cosmetics that include skin-improving ingredients as main ingredients thereof.

The DB management unit 101 may collect reviews written on various web pages through crawling. In other words, the DB management unit 101 may search and crawl the cosmetics sales server, websites, etc. in which reviews are written (e.g., blogs, cafes, pharmaceutical/medical institution websites) to provide reviews of cosmetics selected by users and/or cosmetics recommended by the recommendation algorithm of the server 100.

The DB management unit 101 may analyze reviews among the product information of stored cosmetics, and may filter out and collect formal or false reviews, such as simple sentences (e.g., “I like it”, “I really like it”), sentences including set technical terms, sentences determined to have a high frequency of use of words (e.g., “quality guaranteed”) frequently used in advertisements, sentences containing hyperlinks including phone numbers and email addresses, a plurality of sentences with high similarity, and sentences using preset positive or negative patterns, by using a preset algorithm.

When collecting reviews of cosmetics, the DB management unit 101 may classify and store the reviews on the basis of the number of the reviews, the percentage of positive reviews, ratings, and whether the reviews are formal or false. Alternatively, the DB management unit 101 may classify cosmetics on the basis of the prices of the cosmetics or the purpose/efficacy of the cosmetics.

The skin type determination unit 102 may obtain a facial image of a user captured through a camera (e.g., a camera of the user terminal 200 and/or a camera of the skin measurement device 300). The captured facial image of the user may include distance data. The facial image of the user may be a captured image of a facial region set by AI-based landmarks automatically set on the basis of a distance detection sensor (e.g., a ToF sensor) and/or the camera.

The skin type determination unit 102 may preprocess the facial image by correcting the color values (e.g., RGB pixel values) of the facial image by smoothing colors and removing noise through lighting and color normalization algorithms.

The skin type determination unit 102 may determine landmarks of the facial image of the user captured on the basis of the distance detection sensor and/or the camera. The skin type determination unit 102 may detect the facial region of a user on the basis of the landmarks and determine the skin characteristics of the user on the basis of the detected facial region. The skin characteristics may include a skin type and a skin condition. For example, the skin characteristics may include the moisture, oil, sebum, pH, sensitivity, elasticity, wrinkles, skin color/tone, a pore condition, pigmentation, and dead skin cell condition of the skin. The skin type determination unit 102 may determine the skin characteristics of a user on the basis of a facial image acquired through a separate measuring device (e.g., a measuring mask) other than the user terminal 200 and/or the skin measurement device 300. That is, the skin type determination unit 102 may determine the skin characteristics of a user, such as the moisture, oil, sebum, pH, sensitivity, wrinkles, skin tone, pore condition, pigmentation, and dead skin cell condition of the skin of a user on the basis of the facial image. The skin type of a user may be determined on the basis of the determined skin characteristics.

According to an embodiment, the skin type of a user may be determined by skin condition analysis using Baumann questionnaire, AI-based skin condition analysis, and/or user inquiry responses. The analyzed skin type may be classified into a general skin type, a Baumann skin type, and a Baumann-complemented skin type. The Bauman skin type may be divided into a plurality of skin types by combining oiliness and dryness, sensitivity, wrinkles, pigmentation, etc., and the Baumann-complemented skin type may mean the skin type divided into more diverse types by adding the indicator of inflammation. Here, when classifying skin types, instead of determining the opposite of oiliness as dryness, the opposite of oiliness may be classified as non-oiliness rather than dryness, thereby allowing for a wider variety of skin types.

The skin type determination unit 102 may determine necessary efficacy required for a user depending on the skin type of the user.

The product determination unit 104 may determine first products having the necessary efficacy determined in the DB management unit 101 (e.g., the product DB). The product efficacy determination unit 103 may calculate the efficacy scores of the first products. Since it is impossible to recommend all products with the necessary efficacy in the product DB, the product determination unit 104 may determine the second products with the calculated efficacy scores greater than or equal to a preset efficacy score, among the first products, as products to be recommended to a user.

The product efficacy determination unit 103 may calculate each of the efficacy scores of products in a variety of ways. For example, the product efficacy determination unit 103 may calculate the efficacy scores by determining different weights for ingredients included in products, respectively. For example, the product efficacy determination unit 103 may calculate the efficacy score on the basis of the efficacy of a product claimed (or promoted) by a company of the product, the efficacy of the product determined through review data of the product, etc., in addition to the ingredients required to calculate the efficacy scores.

The product determination unit 104 may determine a predetermined number of products to be recommended for each category classified by purpose of use, such as cleanser, moisturizer, serum, mask, special care, eye care, sunblock, and all-in-one. For example, the product determination unit 104 may determine products to be recommended, such as two cleansers, two moisturizers, three serums, and two sunblocks, depending on the skin type of a user.

In an embodiment, the product determination unit 104 may determine the number of recommended products for each type of products based on the cosmetic usage behavior of a user and the skin type of a user.

In an embodiment, the product determination unit 104 may determine a product that can cover as many diseases as possible depending on the skin type of a user as a recommended product.

In an embodiment, the product determination unit 104 may recommend a generally good product or make a random recommendation when there is no specific disease depending on the skin type of a user.

In an embodiment, when determining products to be recommended to a user, the product determination unit 104 may assign separate identification marks to products that contain low EWG grades, Food and Drug Administration-selected allergens, cautionary ingredients, restricted ingredients, or prohibited ingredients. For example, the product determination unit 104 may assign a first identification mark (e.g., a caution mark) to a product containing hazardous ingredients such as denatured alcohol and fragrance, and conversely, a second identification mark (e.g., recommendation mark) to a product containing ingredients with recommended efficacy for the skin type of a user. The product determination unit 104 may provide a user with information about a reason for each identification mark through the user terminal 200 when assigning the first identification mark and/or the second identification mark.

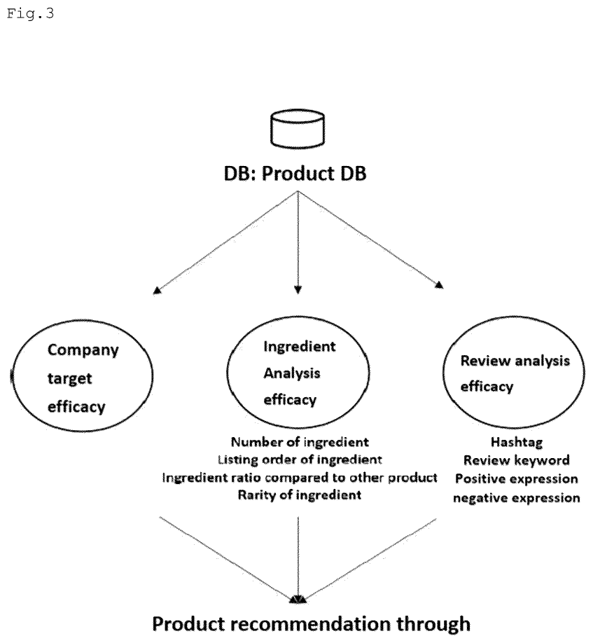

FIG. 3 is a diagram for determining the efficacy of each of products included in the product DB on the basis of at least one of company target efficacy, ingredient analysis efficacy, and review analysis efficacy, and recommending the product to a user.

The product efficacy determination unit 103 may determine the efficacy of a product on the basis of target efficacy of a company that manufactured the product, ingredient analysis efficacy (e.g., description indicated on the product), review analysis efficacy (e.g., keywords extracted from hashtags for the product), etc.

The product determination unit 104 may determine a product to be recommended to a user on the basis of the skin type of the user and the efficacy of ingredients contained in the product. The product efficacy determination unit 103 may determine the positive and negative efficacies of ingredients contained in a product on a user depending on the skin type of the user, and the product determination unit 104 may determine a product to be recommended to the user on the basis of the determined positive and negative efficacies.

In an embodiment, the product efficacy determination unit 103 may determine a weight for each ingredient on the basis of the total number of ingredients contained in a product, an order in which the ingredients are listed on the product, etc. The product efficacy determination unit 103 may calculate the efficacy score for a product (or the main efficacy of the product) by calculating a weighted sum for each ingredient. The product efficacy determination unit 103 may apply lower weights to ingredients with a larger number but listed later in the product, and higher weights to ingredients listed earlier, and may calculate the efficacy score by reflecting the efficacy of the ingredients to which the higher weights are applied.

In an embodiment, the product efficacy determination unit 103 may assign weights to ingredients whose efficacy is unknown or less studied among ingredients listed on the product, as well as the order of the ingredients listed on the product. The product efficacy determination unit 103 may calculate a research level of an ingredient about how extensively the ingredient has been studied on the basis of the number of papers and the number of patents related to the ingredient for each ingredient, and may assign a weight to the ingredient on the basis of the calculated research level. When calculating the research level, the product efficacy determination unit 103 calculates the research level on the basis of the number of overseas papers, the number of domestic papers, the number of overseas patent registrations, the number of overseas patent applications, the number of domestic patent registrations, and the number of domestic patent applications for the ingredient. The research level may be calculated by giving a higher weight in the order of the number of overseas papers, the number of domestic papers, the number of overseas patent registrations, the number of overseas patent applications, the number of domestic patent registrations, and the number of domestic patent applications. The product efficacy determination unit 103 may calculate the efficacy score by assigning a higher weight to an ingredient with a higher research level and a lower weight to an ingredient with a lower research level.

The product efficacy determination unit 103 may obtain ingredient images of each product through the DB management unit 101 or the product DB, and recognize letters and numbers from the obtained ingredient images. The product efficacy determination unit may scan the obtained ingredient images of the product and recognize the letters and numbers included in the ingredient images, respectively. The product efficacy determination unit 103 may extract ingredient information (e.g., the type of ingredients, the number of ingredients, etc.) contained in each of the products on the basis of the recognized letters and numbers.

The product efficacy determination unit 103 may convert the ingredient images of the product into gray scale to effectively extract ingredient information from the ingredient images. The product efficacy determination unit 103 may perform binarization on the ingredient images converted to gray scale, detect a pixel value of each of pixels included in the ingredient images, and determines a first region in which the detected pixel value is lower than a first threshold value and a second region in which the detected pixel value is higher than a second threshold value that is higher than the first threshold value. The product efficacy determination unit 103 may perform image correction to lower the pixel value of the first region by a difference value between the first threshold value and the second threshold value, and to raise the pixel value of the second region by the difference value. The product efficacy determination unit 103 may extract the ingredient information on the basis of ingredient images on which the image correction has been performed.

In an embodiment, the product efficacy determination unit 103 may obtain ingredient information about cosmetics suitable for the determined skin type of a user from the product DB. The product efficacy determination unit 103 may apply a different weight to each ingredient on the basis of the obtained ingredient information and determine the efficacy of a product on the basis of the applied weight.

In an embodiment, when a cosmetic company specifically adds or claims to add a higher amount of a specific ingredient than usual, the product efficacy determination unit 103 may determine the efficacy of the product by giving a higher weight to the specific ingredient. In other words, the product efficacy determination unit 103 may assign a high weight to a target ingredient of a product company for each of the first products included in the product DB.

In an embodiment, the product efficacy determination unit 103 may obtain information about a content ratio for each of ingredients included in each of the first products included in the product DB, and may calculate an average content ratio for each of the ingredients. The product efficacy determination unit 103 may determine the weight of each of the ingredients included in each of the first products on the basis of the content ratio and the average content ratio for each of the ingredients of the first products. The average content ratio may be calculated by summing the content ratios of the first ingredient to the first products that contain the first ingredient and then dividing the sum by the number of the first products that contain the first ingredient.

For example, when general products contain about 5% hyaluronic acid, and the product of a specific cosmetic company contains 20% hyaluronic acid, a higher weighting may be applied to hyaluronic acid.

In an embodiment, in an embodiment, the product efficacy determination unit 103 may crawl ingredients included in each of the first products in the product DB. Among the crawled ingredients, an ingredient (e.g., purified water) used in most products may be assigned a lower weight, and an ingredient used in a small number of products among ingredients included in each of products to be recommended may be assigned a higher weight.

For example, when the number of products including the first ingredient in the product DB is a first number, the number of products including a second ingredient is a second number, and the first number is greater than the second number, the product efficacy determination unit 103 may determine a weight for the second ingredient to be lower than a weight for the first ingredient.

In an embodiment, the product determination unit 104 may determine that when an ingredient included in a product is effective for a disease A, products including the ingredient are effective for the disease A, and may determine the products as products to be recommended to a user. In an embodiment, the product determination unit 104 may determine a product to be recommended to a user on the basis of the efficacy of a product based on correlation between ingredients as well as the efficacy of each of the ingredients as reported in papers, books, and studies.

The product efficacy determination unit 103 may determine a weight for each of ingredients on the basis of a product company target efficacy, ingredient analysis efficacy, and/or review analysis efficacy. For example, the product efficacy determination unit 103 may determine a weight for each of ingredients on the basis of i) the product company target efficacy and the ingredient analysis efficacy, ii) the product company target efficacy and the review analysis efficacy, iii) the ingredient analysis efficacy and the review analysis efficacy, or iv) the product company target efficacy, the ingredient analysis efficacy, and the review analysis efficacy. In other words, the product efficacy determination unit 103 may calculate the efficacy score by comprehensively considering all the indicators described above rather than considering each indicator individually to calculate the efficacy score. The product efficacy determination unit 103 may calculate the efficacy score through the following Mathematical expression 1.

$$S = {\sum }_{a = 1}^{a = m} ( ( {\sum }_{i = 1}^{i = n} {a}_{i} ) \times {w}_{a} ) \qquad [ Mathematical expression 1 ]$$

In Mathematical expression 1 above, S may represent the in efficacy score, a may represent a specific ingredient (e.g., the first ingredient, the second ingredient, . . . , the m-th ingredient), i may represent a weighting indicator for the specific ingredient (e.g., order of notation, a content ratio, a research level, . . . , n), and w may represent correlation between the specific ingredient and the necessary efficacy.

The product efficacy determination unit 103 may calculate the correlation between the specific ingredient and the necessary efficacy on the basis of the number of papers mentioning the specific ingredient and the necessary efficacy, and the number of positive expressions of the specific ingredient for the necessary efficacy in the papers mentioning the specific ingredient and the necessary efficacy.

The product efficacy determination unit 103 may learn a method to determine, as the correct label, target efficacy derived from the review analysis efficacy, the ingredient analysis efficacy, and/or the product company target efficacy, and to assign a weight to each ingredient. The product efficacy determination unit 103 may assign a weight to each ingredient on the basis of the learned result and may calculate the efficacy score of a product on the basis of this.

The product determination unit 104 may recommend cosmetic products suitable for a user on the basis of the calculated efficacy score of the products. The product determination unit 104 may recommend cosmetics suitable for a user on the basis of not only the calculated efficacy score of the product, but also age and preferred brand of each user, climate (season, climate) of an area in which he or she lives, and a skin type thereof. For example, the product determination unit 104 may recommend cosmetic products suitable for a user on the basis of product usage information and product recommendation information of other users who have the same skin type as the user, other users of a similar age as the user, other users who have the same preferred brand as the user, other users whose region of residence has climate similar to the climate of the residence of the user, and/or other users according to a combination thereof.

The recommendation list provision unit 105 may generate a recommendation list of cosmetics for a user on the basis of the determined cosmetics. The recommendation list provision unit 105 may transmit information about the generated recommendation list and cosmetics included in the recommendation list to the user terminal 200. When generating the recommendation list, the recommendation list provision unit 105 may indicate a product containing ingredients that have a negative effect on the skin type of a user with an identification label (e.g., a red boundary).

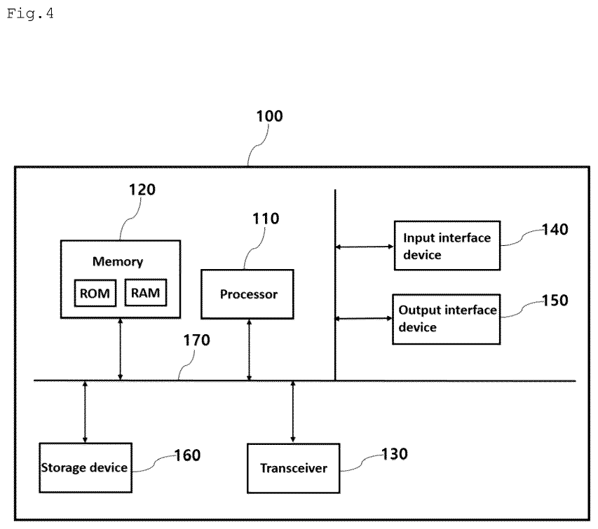

FIG. 4 is a diagram illustrating the configuration of the hardware of the product recommendation server 100 according to FIG. 1.

Referring to FIG. 4, the product recommendation server 100 may include at least one processor 110 and a memory that stores instructions directing the at least one processor 110 to perform at least one operation.

The at least one operation may include at least some of the operations or functions of the server 100 described above and may be implemented in the form of instructions and performed by the processor 110.

Here, the at least one processor 110 may refer to a central processing unit (CPU), a graphics processing unit (GPU), or a dedicated processor on which methods according to the embodiments of the present disclosure are performed. Each of a memory 120 and a storage device 160 may be configured as at least one of a volatile storage medium and a non-volatile storage medium. For example, the memory 120 may be one of a read only memory (ROM) and a random access memory (RAM), and the storage device 160 may be a flash memory, a hard disk drive (HDD), a solid state drive (SSD), or various memory cards (e.g., a micro SD card), etc.

Additionally, the server 100 may include a transceiver 130 that performs communication via a wireless network. In addition, the server 100 may further include an input interface device 140, an output interface device 150, the storage device 160, etc. Each component included in the server 100 may be connected to each other by a bus 170 to communicate with each other. FIG. 4 illustrates the product recommendation server 100 as an example, but is not limited thereto. For example, a plurality of user terminals may include the components of FIG. 4.

The methods according to the present disclosure may be implemented in the form of program instructions that can be executed through various computer means and may be recorded in a computer-readable medium. The computer-readable medium may include program instructions, data files, data structures, etc., either alone or in combination. The program instructions recorded in the computer-readable medium may be specially designed and configured for the present disclosure or may be known and available to those skilled in the art of computer software.

Examples of computer-readable media may include a hardware device specifically configured to store and execute program instructions, such as ROM, RAM, flash memory, and the like. Examples of the program instructions may include high-level language codes that can be executed by a computer by using an interpreter, etc. as well as machine language codes, such as those produced by a compiler. The hardware device described above may be configured to operate with at least one software module to perform the operations of the present disclosure, and vice versa.

In addition, the above-described method or device may be implemented by combining all or part of configuration or function thereof, or may be implemented by separating them.

Although the present disclosure has been described above with reference to preferred embodiments thereof, it will be understood by those skilled in the art that various modifications and changes may be made to the present disclosure without departing from the spirit and scope of the present disclosure as set forth in the claims below.

1. A product recommendation server for recommending a product suitable for a user, the server comprising:
a skin type determination unit that obtains a facial image of a user and determines a skin type of the user on the basis of the obtained facial image;
a product efficacy determination unit that determines product efficacy of each of products comprised in a product DB;
a product determination unit that determines a product to be recommended to the user on the basis of the determined skin type and the determined product efficacy; and
a recommendation list provision unit that generates a recommendation list for the user on the basis of the determined product.

2. The server of claim 1, wherein the skin type determination unit determines necessary efficacy required for the user on the basis of the determined skin type,
the product determination unit determines first products having the necessary efficacy from the product DB, and
the product efficacy determination unit calculates an efficacy score of each of the first products.

3. The server of claim 2, wherein the product efficacy determination unit
recognizes letters in ingredient images of each of the products,
extracts ingredient information comprised in each of the products on the basis of the recognized letters,
determines a weight for each of ingredients on the basis of types of the ingredients comprised in each of the products, order of listing of the ingredients listed in each of the products, and the number of the listed ingredients, and
calculates the efficacy score on the basis of the weight for each of the ingredients.

4. The server of claim 3, wherein the product determination unit determines second products with a preset score or higher among the first products as products to be recommended to the user on the basis of the calculated efficacy score of each of the products.

5. The server of claim 1, wherein the product efficacy determination unit determines the efficacy of each of the products on the basis of at least one of target efficacy targeted by a product company, ingredient efficacy based on ingredient information, and efficacy based on review analysis.
